(19) United States
(12) Patent Application Publication
(10) Pub. No.: US 2019/0160109 A1
(43) Pub. Date: May 30, 2019

(54) Notochordal cell matrix as a bioactive lubricant for the osteoarthritic joint

(71) Applicant: Technische Universiteit Eindhoven, Eindhoven (NL)

(72) Inventors: Keita Ito, Helmond (NL); Stefan Antonius Henricus de Vries, Veldhoven (NL)

(21) Appl. No.: 16/261,771

(22) Filed: Jan. 30, 2019

Related U.S. Application Data
(63) Continuation-in-part of application No. 16/068,752, filed on Jul. 9, 2018.
(60) Provisional application No. 62/623,672, filed on Jan. 30, 2018.
(60) Provisional application No. 62/277,032, filed on Jan. 11, 2016.

(51) Int. Cl. A61K 35/32; A61K 9/19; A61K 9/08; A61K 9/06

(52) U.S. Cl. CPC A61K 35/32; A61K 9/06; A61K 9/08; A61K 9/19

(57) ABSTRACT

A solubilized notochordal cell matrix powder dissolved in a carrier solvent or formed as a gel is provided. The notochordal cell matrix powder originates from lyophilized and treated porcine nucleus pulposus tissue containing notochordal cells. The powder contains less than 20% of porcine nucleid acids, and the powder contains a substantially unchanged amount of porcine protein content compared to the originating porcine nucleus pulposus tissue. The solubilized notochordal cell matrix powder is capable of stimulating native or stem cells to proliferate and produce a significant increase in glycosaminoglycans and type-II collagen matrix. Embodiments of the invention can be used as a lubricant with regenerative potential for application in the osteoarthritic joint.

### CROSS-REFERENCE TO RELATED APPLICATIONS

This application claims priority from U.S. Provisional Patent Application 62/623,672 filed Jan. 30, 2018, which is incorporated herein by reference.

This application is a continuation-in-part of U.S. patent application Ser. No. 16/068,752 filed Jul. 9, 2018, which is incorporated herein by reference.

U.S. patent application Ser. No. 16/068,752 is a 371 of PCT/EP2017/050431 filed Jan. 11, 2017. PCT/EP2017/050431 claims the benefit of U.S. Provisional Application 62/277,032 filed on Jan. 11, 2016.

### FIELD OF THE INVENTION

This invention relates to methods, structures and compositions using a notochordal cell matrix as a bioactive lubricant.

### BACKGROUND OF THE INVENTION

Articular cartilage (AC) is a layer of smooth hydrated tissue that covers the articulating surfaces of bones in fluid filled synovial joints. Together with the synovial fluid, it provides low friction in these joints during motion. Osteoarthritis (OA), a degenerative joint disease, affects the AC as well as the synovium and subchondral bone, leading to painful articulating dysfunction. Knee OA is one of the leading causes for pain and disability worldwide, with estimates suggesting 9.3 million affected people in the US alone.

Osteoarthritis is initially treated conservatively, with exercise and pain medication. In later stages, non-steroidal anti-inflammatory drugs or intra-articular steroid injections are prescribed. Another treatment option is viscosupplementation i.e. intra-articular injection of hyaluronic acid (HA), a large polysaccharide that is naturally found in synovial fluid. Hyaluronic acid increases the viscosity of the synovial fluid in addition to providing viscoelasticity thereby contributing to hydrodynamic lubrication of the joint. Furthermore, due to molecular interactions at the cartilage surface, it contributes to boundary lubrication as well. With OA, HA degrades resulting in a decreased concentration and low molecular weight fragments, which affects the lubricating properties of synovial fluid. Injection of HA into the joint aims to increase synovial fluid viscosity and minimize pain to postpone total knee replacement. Although meta-analyses provide contradicting results regarding the efficacy of HA viscosupplementation, it is generally considered as a safe and effective treatment for painful knee OA. Despite HA's positive effects, it only provides symptomatic relief and does not restore the affected cartilage to a healthy state. Therefore, other options should be explored. This invention explores the option of a notochordal cell matrix as a bioactive lubricant for the osteoarthritic joint.

### SUMMARY OF THE INVENTION

The present invention provides a method of making a notochordal cell matrix solution or gel for the purposes of regenerating the intervertebral disc or as a bioactive lubricant for an osteoarthritic joint. In addition, the invention provides an intervertebral disc regeneration stimulant or bioactive lubricant processed by the method as described.

In one embodiment, first, porcine nucleus pulposus tissue containing notochordal cells is lyophilized to destroy cells within the tissue and to make a dry and brittle tissue. Second, the dry and brittle tissue is treated by DNAse, RNAse, detergent, or a combination thereof (e.g. benzonase & Triton) to remove cellular and nucleic acid remnants. This treatment results in at least 80% removal of porcine nucleic acids from the porcine nucleus pulposus tissue, while substantially maintaining porcine protein content within the porcine nucleus pulposus tissue. In a different way of saying, the treatment results in the material containing less than 20% of porcine nucleic acid, and containing a substantially unchanged amount of porcine protein content compared to the originating porcine nucleus pulposus tissue.

The order of first lyophilization followed by nucleic acid removal is important to achieve this result. Porcine genomic material harbors endogenous retroviruses (PERVs). PERVs do not come to expression in the pig itself, but may become active upon implantation in different species, e.g. human. Hence, removal of genomic material to minimize the risk of disease transmission is an important step prior to its application in a regenerative therapy.

In the case of removal of nucleic acids from NP tissue, either in a wet state or after disintegration, which is different from the present invention, such treatment has resulted in the removal of more than 90% of the nucleic acid content but also at a considerable loss of extracellular matrix proteins (>40%). The present invention teaches the substantial removal of nucleic acid from porcine NP tissue, while largely or substantially maintaining its protein content (e.g. active factors).

Third, the treated material is further lyophilized and pulverized into a notochordal cell matrix powder. The notochordal cell matrix powder is solubized by dissolving the notochordal cell matrix powder in a carrier solvent or formed as a gel.

The solubilized notochordal cell matrix powder is capable of stimulating native or stem cells to proliferate and produce a substantial increase in glycosaminoglycans and type-II collagen matrix.

The solubilized notochordal cell matrix powder is capable of stimulating native or stem cells to proliferate and produce a significant increase in glycosaminoglycans. In one example, the solubilized notochordal cell matrix powder is capable of stimulating native or stem cells to proliferate and produce an at least a multi-fold (e.g. at least two to three times) increase in glycosaminoglycans. In another example, the solubilized notochordal cell matrix powder is capable of stimulating native or stem cells to proliferate and produce an at least a seven-times increase in glycosaminoglycans. In general, the increase depends on the species, the dosage of powder administered and whether it is administered in-vitro or in in-vivo.

The intervertebral disc regeneration stimulant solution or gel is defined as a solubilized notochordal cell matrix powder dissolved in a carrier solvent or formed as a gel, whereby the notochordal cell matrix powder originates from lyophilized and treated porcine nucleus pulposus tissue containing notochordal cells. The powder contains less than 20% of porcine nucleic acid, and the powder contains a substantially unchanged amount of porcine protein content compared to the originating porcine nucleus pulposus tissue.

Embodiments of the invention have the following advantages:
Porcine notochordal nucleus puposus tissue is plentiful.
Porcine notochordal nucleus puposus tissue can be easily processed following the described steps, supra, to produce a powder that is not harmful and which can stimulate IVD regeneration.
By using notochordal cell matrix powder, the difficult and time-consuming step to identify and produce the specific soluble active factors secreted by notochordal cells is unnecessary.
The solubilized notochordal cell matrix powder can be injected into the IVD through a small gage needle (<27G) causing minimal harm to its outer annulus fibrosus.
The solubilized notochordal cell matrix powder forms a self-assembling hydrogel at higher concentrations, may be used as a (stem) cell carrier, to replenish the decreasing cell number that characterizes intervertebral disc degeneration.
The solubilized notochordal cell matrix powder is sufficient, on its own, to induce stem cells to become NPCs.
The solubilized notochordal cell matrix powder at higher concentrations has rheological properties similar to the natural nucleus pulposus. Thus, it can replenish and easily integrate into the nucleus pulposus tissue.
The solubilized notochordal cell matrix powder at higher concentrations has considerable swelling potential. Thus, it can add swelling pressure to the nucleus pulposus.
The solubilized notochordal cell matrix powder can be combined with suitable carriers, creating a sustained release of its soluble components and therefore a longer-lasting regenerative effect.

In addition, the invention further provides that the notochordal cell matrix (NCM) has regenerative potential on nucleus pulposus cells of the intervertebral disc in vitro and in vivo. It has also been demonstrated that NCs secrete factors with anabolic and anti-inflammatory potential on human chondrocytes. Moreover, when dissolved at a low concentration, NCM forms a viscous fluid that may have lubricating properties. Therefore, this invention describes a novel approach to use NCM as a regenerative lubricant for application in the osteoarthritic (OA) joint. In summary and in support of this claim, bovine chondrocyte-seeded alginate beads were cultured in medium supplemented with NCM to test NCM's regenerative potential. In addition, alginate beads were also cultured in NCM stimulated with IL-1β, to investigate NCM's effects in an inflammatory environment. Lastly, reciprocating sliding friction tests of cartilage on glass were performed to test NCM's lubricating properties relative to and in combination with hyaluronic acid (HA). NCM increased GAG deposition and cell proliferation, as well as GAG per DNA ratio and hydroxyproline content. These effects were maintained in the presence of IL-1β. NCM also mitigated expression of IL-1β-induced IL-6, IL-8, ADAMTS-5 and MMP-13. Furthermore, NCM induced a dose-dependent reduction of the coefficient of friction (CoF) similar to HA at a test speed of 6, as well as 60 mm/s. The results from this invention indicate that NCM has anabolic and anti-inflammatory effects on chondrocytes, as well as favorable lubricating properties. Therefore, intra-articular NCM injection have potential as a treatment to minimize pain while restoring the affected cartilage tissue in the OA joint, and warrants further investigation.

### BRIEF DESCRIPTION OF THE DRAWINGS

FIGS. 1A-C shows according to exemplary embodiments of the invention the biochemical content expressed as glycosaminoglycan (GAG) in FIG. 1A, DNA in FIG. 1B, and GAG per DNA in FIG. 1C of NPCs embedded in alginate beads, treated with base medium (BM), base medium plus soluble factors from medium conditioned with notochordal cell tissue culture (NCCM), or base medium plus solubilized notochordal cell matrix powder (NCM) in culture for 4 weeks. NCM not only stimulates cells to proliferate, but also each cell to produce more GAG resulting in almost a 7× overall increase in GAG production.

FIG. 2 shows according to exemplary embodiments of the invention alcian blue staining of base medium (BM), base medium plus soluble factors from medium conditioned with notochordal cell tissue culture (NCCM), or base medium plus solubilized notochordal cell matrix powder (NCM) treated NPC embedded alginate beads cultured for 4 weeks, at 10× and 40× magnification. Darker homogenous staining of NCM exposed beads indicated increased and well distributed GAG production. The increased proliferation of cells in clusters with NCM is also demonstrated.

FIG. 3 shows according to exemplary embodiments of the invention collagen type I and type II immunohistochemical staining of NPCs embedded in alginate beads, treated with base medium (BM), base medium plus soluble factors from medium conditioned with notochordal cell tissue culture (NCCM), or base medium plus solubilized notochordal cell matrix powder (NCM) in culture for 4 weeks. As indicated by darker staining, NCM hardly induce any production of type I collagen but was a very strong stimulant for type II collagen.

FIGS. 4A-B shows according to exemplary embodiments of the invention in FIG. 4A DNA and in FIG. 4B protein content in untreated and benzonase-treated solubilized porcine notochordal cell matrix powder, where “hr” denotes exposure duration to benzonase and “U” to the benzonase concentration in IU/ml. After treatment for 6 hr with 100 or 200 IU/ml benzonase only 20% of the porcine DNA remained in the NCM while the majority of the protein remained.

FIG. 5 shows according to an exemplary embodiment of the invention as an instructive stem cell carrier. The left panel is a live/dead cell staining with calcein-AM and propridium iodide of human bone marrow stromal cells (MSCs) mixed in 20% w/vol solubilized notochordal cell matrix powder, injected through a 27G needle and subsequently cultured in base medium (24 hrs). The right panel are Safranin O stained thin histology sections of MSC pellet cultures (4 weeks) in base medium (control), base medium plus 10 ng/ml TGFβ1 (+TGFβ1) or base medium plus 10 mg/ml solubilized notochordal cell matrix powder (+NCM). After injection in NCM, MSCs remain viable (solid & hollow arrows). Within 24 hr, they attach to the NCM forming a more spindle shape (hollow arrows). NCM stimulates MSC to proliferate (larger cell mass) and assume a chondrogenic phenotype producing a matrix rich in GAGs (Safranin O=lighter stain).

FIGS. 6A-C show according to an exemplary embodiment of the invention the rheology and osmotic properties of the solubilized notochordal cell matrix powder: FIG. 6A) storage (G′) and loss (G″) modulus and FIG. 6B) phase shift (tan δ) frequency sweep of bovine young adult nucleus pulposus tissue (NP) and 10, 20 and 30% w/vol solubilized notochordal cell matrix powder (NCM); FIG. 6C) osmotic pressure as function of solubilized notochordal cell matrix powder concentration (% w/vol, NCM gel) calculated from swelling against various concentration polyethylene glycol solutions (analysis method as outlined in NO Chahine, et al. Biophys J, 89(3):1543-1550, 2005). The rheological properties of NCM, especially at higher concentrations approach that of natural nucleus pulposus tissue. At concentrations greater than 15% w/vol, NCM has enough osmotic potential to restore tonicity to nucleus pulposus tissue whose own natural osmotic potential is 0.1-0.2 MPa.

FIGS. 7A-E show according to an exemplary embodiment of the invention porcine NC-rich NP matrix (NCM) induced an anabolic response of bovine chondrocytes (FIG. 7A) Glycosaminoglycan (GAG) and (FIG. 7B) DNA content per alginate bead seeded with bovine chondrocytes, (FIG. 7C) GAG per DNA and (FIG. 7D) hydroxyproline content per bead. Values represent means+standard deviations, n=5 per group. * indicates p<0.05 compared to all other groups, # indicates p<0.05 compared to base medium (BM). (e) Alcian blue staining confirms increased GAG deposition with NCM and BM supplemented with 10 ng/ml TGF-β1 (TGF) compared to BM. Collagen immunohistochemistry shows increased collagen type II at the edge of the bead and more diffuse collagen type II deposition with TGF. Collagen type I staining intensity appears to be increased with NCM.

FIG. 8 shows according to an exemplary embodiment of the invention NC-rich nucleus pulposus matrix (NCM) has distinct anabolic effects in chondrocyte-seeded alginate beads. ACAN: aggrecan; COL-2: collagen type II alpha 1; COL-1: collagen type I alpha 1. Expression levels are relative to 60S ribosomal protein L13 (RPL13). Values are means+standard deviations, n=5 per group. * indicates p<0.05 compared to all other groups at the same time point, # indicates p<0.05 compared to base medium (BM).

FIGS. 9A-E show according to an exemplary embodiment of the invention that addition of an inflammatory stimulus did not affect NCM's regenerative potential (FIG. 9A) Glycosaminoglycan (GAG) and (FIG. 9B) DNA content per alginate bead seeded with bovine chondrocytes, (FIG. 9C) GAG per DNA and (FIG. 9D) hydroxyproline content per bead. Values represent means+standard deviations, n=5 per group. # indicates p<0.05 from both base medium (BM) groups. (FIG. 9E) Alcian blue staining confirms increased GAG deposition with both NCM groups compared to both BM groups. Collagen type II staining was less intense with addition of IL-1β to BM relative to BM alone which, albeit to a lesser extent, is also observed with addition of IL-1β to NCM. Collagen type I deposition appeared to increase with addition of IL-1β to BM, though this is not observed with addition of IL-1β to NCM.

FIG. 10 shows according to an exemplary embodiment of the invention that NC-rich nucleus pulposus matrix (NCM) may have anti-inflammatory and -catabolic potential. IL-1β/6/8: interleukin-1β/6/8; TNFα: tumor necrosis factor α; ADAMTS-5: a disintegrin and metalloproteinase with thrombospondin motifs 5; MMP-13: matrix metalloproteinase 13; ACAN: aggrecan; COL-2: collagen type II alpha 1. COL-1: collagen type I alpha 1. Expression levels are relative to 60S ribosomal protein L13 (RPL13). Values are means+standard deviations, n=4-5 per group. * indicates p<0.05 compared to all other groups from the same time point, $ indicates p<0.05 compared to base medium (BM), # indicates p<0.05 compared to BM+IL-1β.

FIGS. 11A-B show according to an exemplary embodiment of the invention that NC-rich nucleus pulposus matrix (NCM) has potential in cartilage lubrication. Coefficients of friction (COF) at cycle 20 in the different lubricants normalized to COF at cycle 20 in PBS alone at (FIG. 11A) 6 mm/s and (FIG. 11B) 60 mm/s. BSA: 5 mg/ml bovine serum albumin, HA: 4 mg/ml hyaluronic acid, NCMl: 4 mg/ml NCM, NCMh: 10 mg/ml NCM. Values are mean+standard error, n=5 for 6 mm/s measurements, n=3 for 60 mm/s measurements. * indicates p<0.05 compared to BSA, # indicates p<0.05 compared to BSA+HA, $ indicates p<0.05 compared to BSA+NCMl.

FIGS. 12A-B show according to an exemplary embodiment of the invention FIGS. 12A-B: Repeated rounds of reciprocating sliding of cartilage against glass in PBS do not affect coefficients of friction (CoF). CoF for each cycle of 4 consecutive rounds (PBS1-PBS4, n=4) of cartilage on glass sliding at (FIG. 12A) 6 mm/s and (FIG. 12B) 60 mm/s. Significant differences were observed between multiple rounds of sliding at either of the test speeds for individual measurements.

### DETAILED DESCRIPTION

According to an embodiment of the invention, healthy NC-rich NP tissue is harvested, e.g. from porcine spines. The tissue is then lyophilized, thereby destroying the cells, and leaving behind a dry brittle tissue. Benzonase, DNAse, RNAse, detergent, or a combination thereof is then used to remove the cellular and nucleic acid remnants, after which the tissue is lyophilized again. The brittle tissue is then pulverized to a fine powder. This powder can be dissolved in a carrier solvent like PBS, a viscous carrier like hyaluronic acid or even a sustained release hydrogel or microspheres, and injected into the IVD to stimulate intervertebral disc regeneration. According to the invention, the dissolved and directly injected powder has the ability to promote healthy matrix production and inhibit inflammation. Additionally, it may inhibit matrix catabolism, inhibit neovascularization and inhibit neo-innervation within the IVD.

### Exemplary Embodiment and Results

An experiment was performed to test whether the powdered NC-rich NP tissue Matrix (NCM) has a stimulatory effect and how similar this is to just proteins produced by NC cells, i.e. NC Conditioned Medium (NCCM).

NCCM was produced by incubating porcine NC-rich NP tissue for 4 days in high glucose DMEM with 1% penicillin/streptomycin (p/s) at 37° C., 5% CO₂ and 5% O₂. Afterwards, the medium was poured through a 70 μm strainer to remove the NCs and tissue. Subsequently, it was filtered through a 3 kDa filter, the solutes that remained where resuspended in low glucose (lg) DMEM, and the NCCM was stored at −80° C.

NCM was produced by lyophilizing porcine NP tissue overnight after which it was ground to a fine powder. The protein content of both NCCM and NCM was measured so that when NCM was resuspended in low glucose (lg) DMEM, the final protein concentrations of both were the same.

These (NCM and NCCM) were compared to base medium (BM=lgDMEM supplemented with ITS, ascorbic acid, L-proline, bovine serum albumin and p/s).

Cells, similar to those in human adult discs, i.e. NPCs, were harvested from the caudal discs of bovine tails by enzymatic digestion, and seeded in 1.2% alginate beads at 3 million cells/ml alginate. The beads were cultured for 4 weeks in BM, NCCM or NCM (both with the same supplements as BM).

After culture, the beads were analysed for glycosaminoglycan (GAG) and DNA content (directly correlated to cell number). GAG is the main ECM component of NP tissue, it gives the NP tissue its functional mechanical properties and it is the first to be decreased during IVD degeneration.

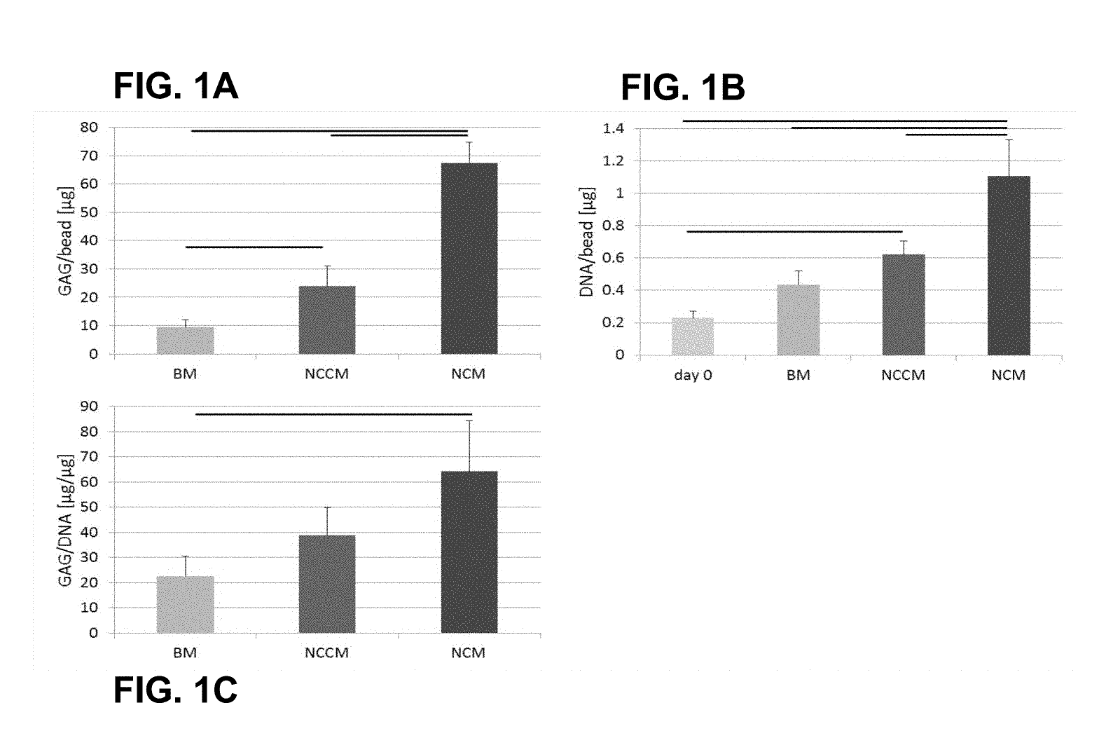

As observed previously, just the proteins derived from NCs (NCCM) caused the disc cells to proliferate 1.3× compared to BM (FIG. 1B). It also stimulated each cell to produce 1.8× more GAG than those cultured without the added proteins (BM, FIG. 1C). These two mechanisms combined resulted in an overall increase in GAG content that was a little over 2× with the NC proteins (NCCM) than without (BM, FIG. 1A).

With the powdered NC-matrix added to the medium (NCM), the cells proliferated almost 5× more than without (BM, FIG. 1B), and stimulated each cell to produce 3× more GAG (FIG. 1C). Thus, the overall increase in stimulated GAG content was 7× greater with the NC-matrix powder than without (NCM vs BM, FIG. 1A). This superior stimulation of GAG production by NP-matrix powder (NCM) as compared to just the proteins from NCs (NCCM) was unexpected and surprising, suggesting that either presentation of the NC-derived proteins or a synergistic effect combining the proteins with the NC-matrix provides a greater anabolic effect on cells in the center of our discs.

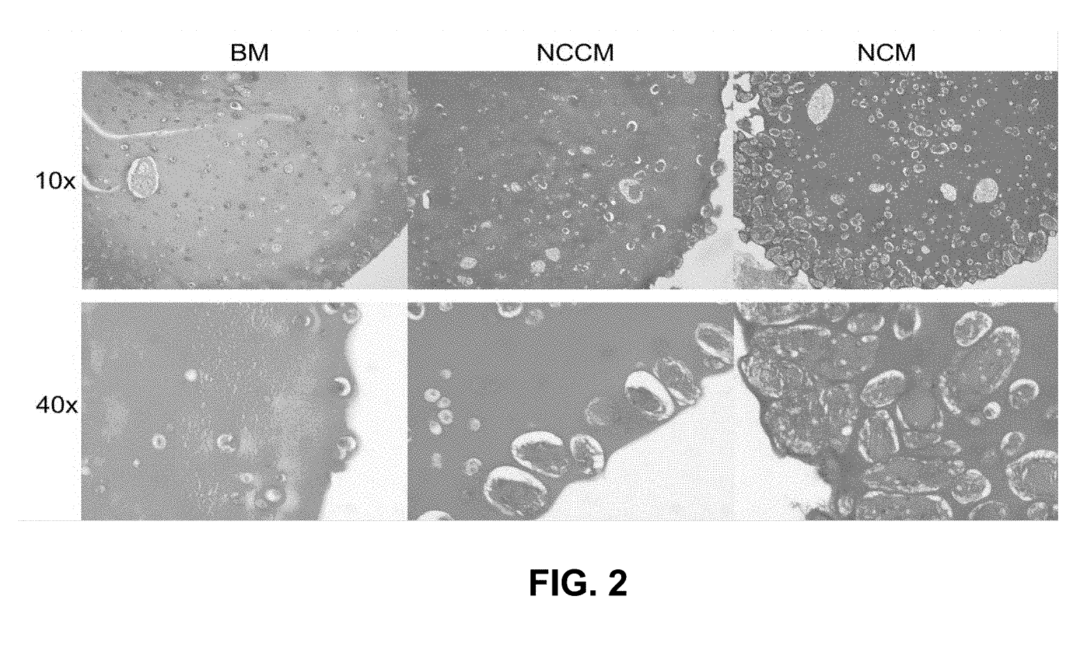

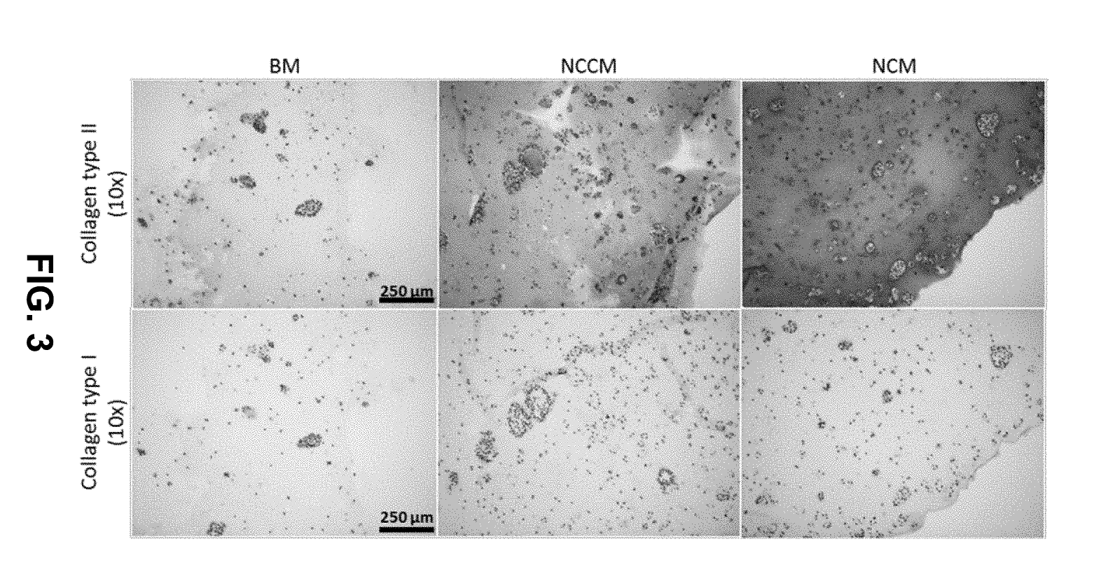

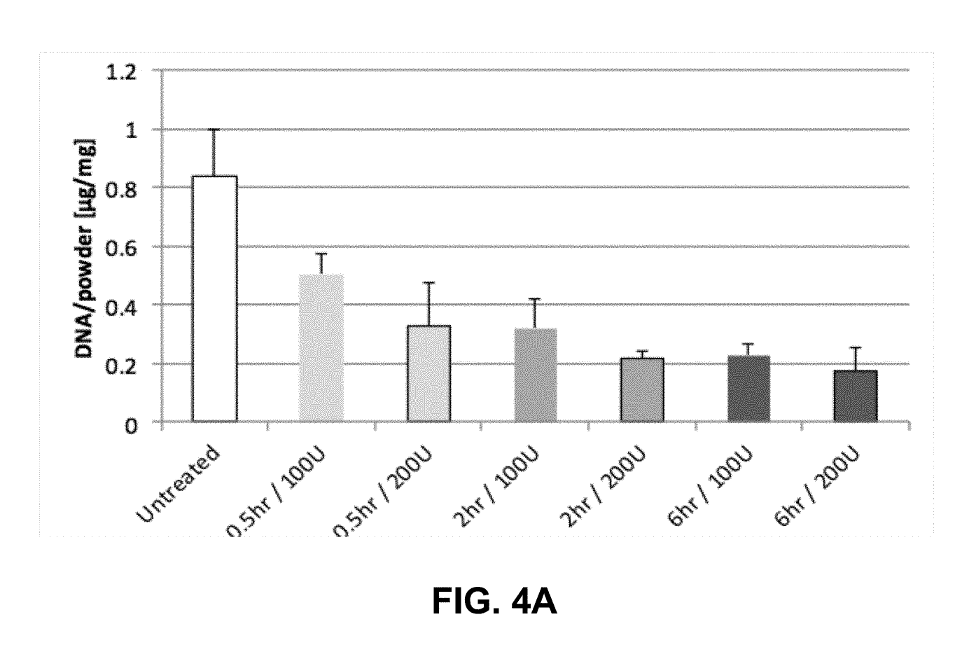

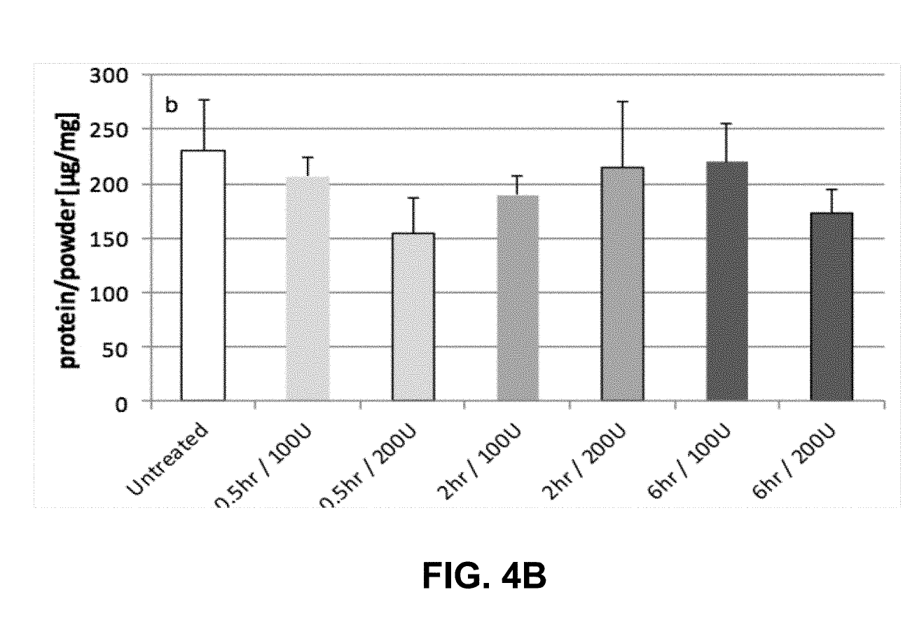

The results from the biochemical assays were also confirmed histologically on Alcian blue stained sections (FIG. 2), where darker blue in NCCM and especially NCM indicates a higher GAG content. At 40× magnification, an increased cell number is observed in NCM compared to NCCM and especially BM.

In vitro experiments have been conducted to further assess the feasibility of NC-matrix powder in intervertebral disc regeneration. NCCM has been demonstrated to have anti-some catabolic and anti-inflammatory effect on inflammation-induced NPCs. Furthermore, NCCM has been shown to inhibit neurite growth and blood vessel formation. We expect the NCM to have similar or better effects than NCCM.

Pigs harbor endogenous retroviruses (PERVs) in their genome, which are able to infect human cells. Therefore, before clinical application becomes feasible, NC-matrix powder (NCM) needs to be decellularized, while maintaining a high as possible protein content. Decellularization is performed with benzonase, which cleaves DNA and RNA to small fragments that can afterwards be removed through washing steps.

A test was performed to remove DNA from NCM. Again, NCM was produced by lyophilizing and grinding porcine NC-rich NP tissue (n=3). The powder was then incubated on a shaker at 37° C. with 2 different concentrations of benzonase (100 U/ml and 200 U/ml) in Tris-EDTA buffer, up to 3 different timepoints (30 min, 2 hrs or 6 hrs). Afterwards, the samples were washed twice with PBS, and the untreated powder, as well as each of the treatment groups, was assayed for DNA and protein content. DNA content decreased with duration of benzonase treatment, and small differences were observed between the two benzonase concentrations. Although some variations in protein content were observed between treatment groups, there were no clear trends for decreasing protein content with treatment time or benzonase concentration. Altogether, these results indicate that it is possible to remove DNA from the NCM, while largely maintaining the protein content.

Once the effects of NC-matrix powder on vessel- and neurite formation, as well as its anti-inflammatory effects have been established, in vivo animal experiments will be performed. If successful, NC-matrix powder could be tested in clinical trials.

In vitro experiments with bovine NPCs used ˜2 mg NCM/6*10⁵ NPCs. This resulted in a strong increase in GAG production as well as cell proliferation. Based on work with Beagle's thoracic NP tissue and bovine caudal NP tissue, we expect the weight of the Beagle's lumbar NP tissue to be ˜100 mg, containing ˜2*10⁵ NPCs. Hence, the NCM dosage range that will therefore be tested in a canine in vivo model will be 1 mg, 5 mg, 10 mg and 20 mg NCM per 100 mg NP tissue. Because bio-availability will be affected by the tissue, organ and injection method and cell response in vivo may be different to in vitro, a broad dosage range should be explored. When translating from the canine to human, the exact interspecies differences are also unknown and we speculate that dosages for the human may be +/−one order of magnitude different. For example these could be as broad as 0.1 to 100 mg NCM powder to every 100 mg NP tissue of the human disc.

Embodiments of the invention can be used for the disc regenerative treatment of discogenic back and neck pain in an orthopaedic and/or pharmaceutical setting/approach.

In another embodiment, the notochordal cell matrix (NCM) is a hydrogel. In a concentrated form, 10 to 30% w/vol, the NCM behaves like a loose self-assembling hydrogel that can be injected through a hypodermic needle.

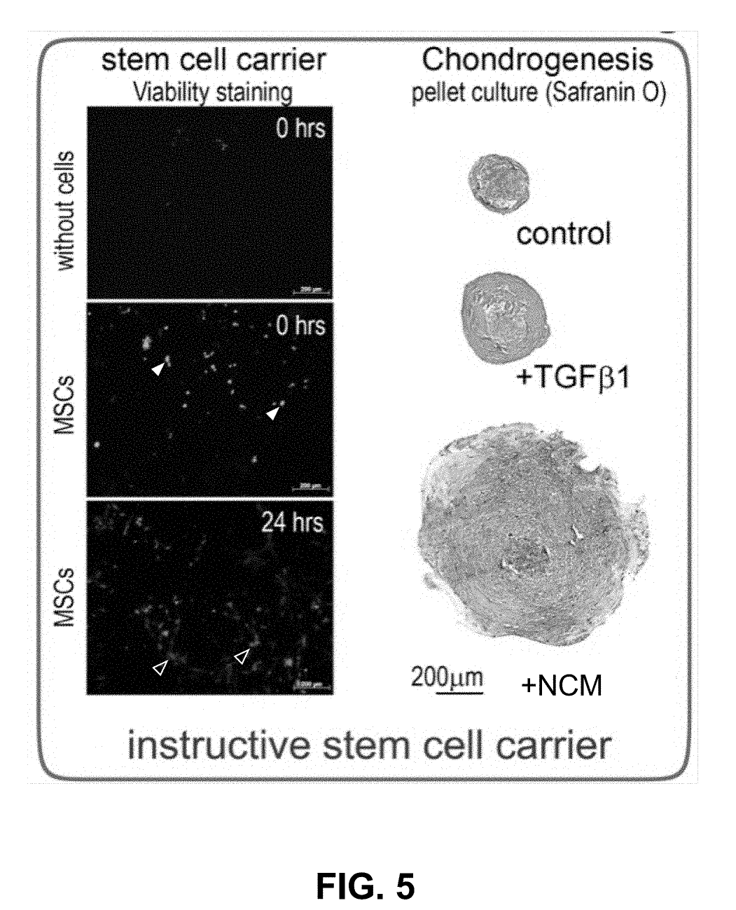

In yet another embodiment, the NCM can be used as a cell carrier. When mixed and injected through a 27G needle, human bone marrow stromal cells (hBMSCs) maintain their viability (0 hr, rounded form) and thereafter even attach to the matrix components of the NCM hydrogel (spindle form, FIG. 5).

In still another embodiment, the NCM can be progenitor cell instructive. When progenitor cells (hBMSCs) in pellet culture are exposed to NCM (10 mg/ml) in a 4 week culture, they are instructed to differentiate into chondrogenic cells producing a matrix rich in proteoglycans and collagen type-II (FIG. 5).

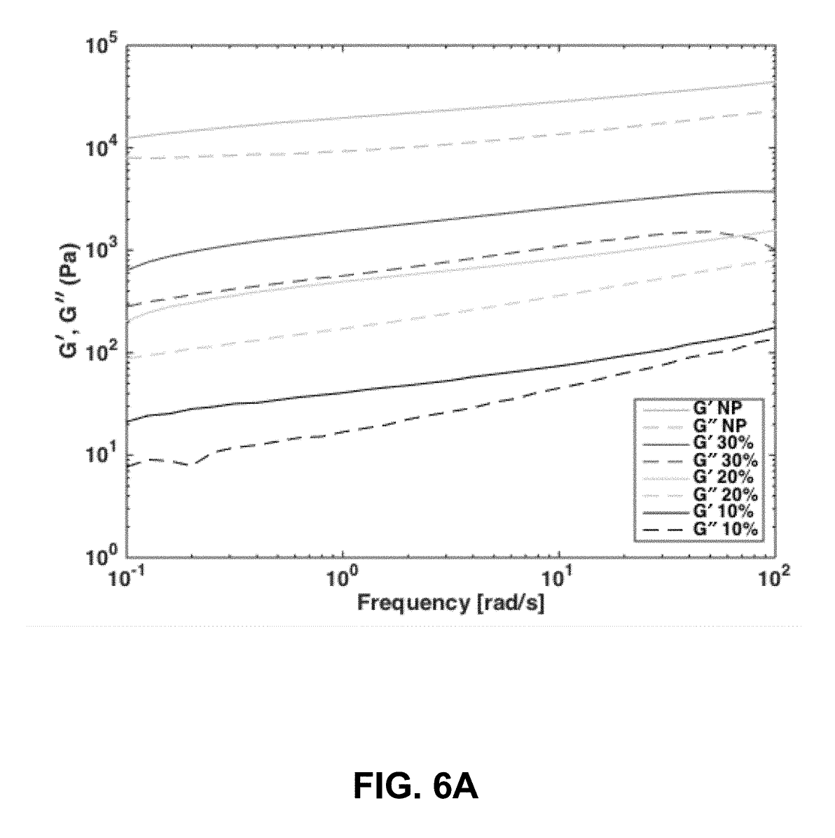

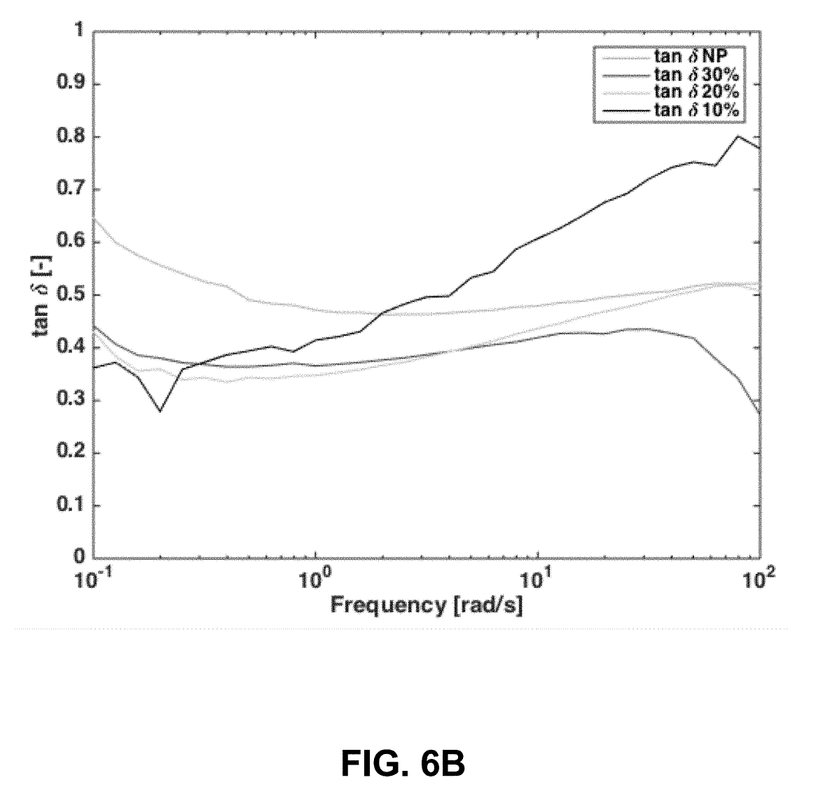

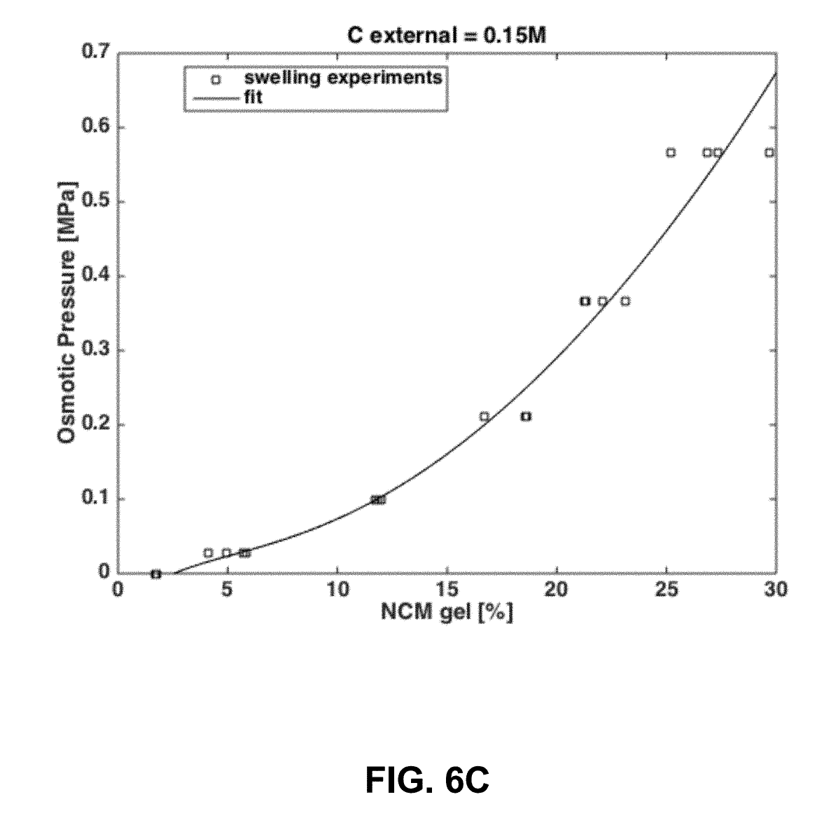

In still another embodiment, the NCM has material properties, which can help to restore the biomechanical characteristic of the IVD to a healthy state. The NCM has rheological properties that are concentration dependent and that at higher concentrations start to approach that of the natural NP tissue (FIGS. 6A-B). NCM also has considerable osmotic swelling potential (FIG. 6C), which can help to directly restore the swelling properties of glycosaminogycan (GAG) depleted moderately degenerated discs.

In an additional embodiment, the use of NCM as a biomaterial with lubricating properties is provided, that could simultaneously stimulate chondrocytes to restore the affected cartilage within the OA joint. Towards this embodiment, first, the regenerative potential of NCM on bovine chondrocytes was investigated in an in vitro alginate bead culture. Second, it was investigated whether NCM could also stimulate chondrocytes in the presence of an inflammatory stimulus. Lastly, reciprocating sliding cartilage on glass friction tests were performed to test NCM's lubricating properties relative to and in combination with hyaluronic acid (HA). For specific details on materials and methods towards this additional embodiment, different from the methods and materials described infra, the reader is referred to U.S. Provisional Patent Application 62/623,672 filed Jan. 30, 2018, which is incorporated herein by reference. The results providing additional support towards the embodiment of the use of NCM as a biomaterial with lubricating properties that could simultaneously stimulate chondrocytes to restore the affected cartilage within the OA joint are described infra.

### NCM's Regenerative Potential

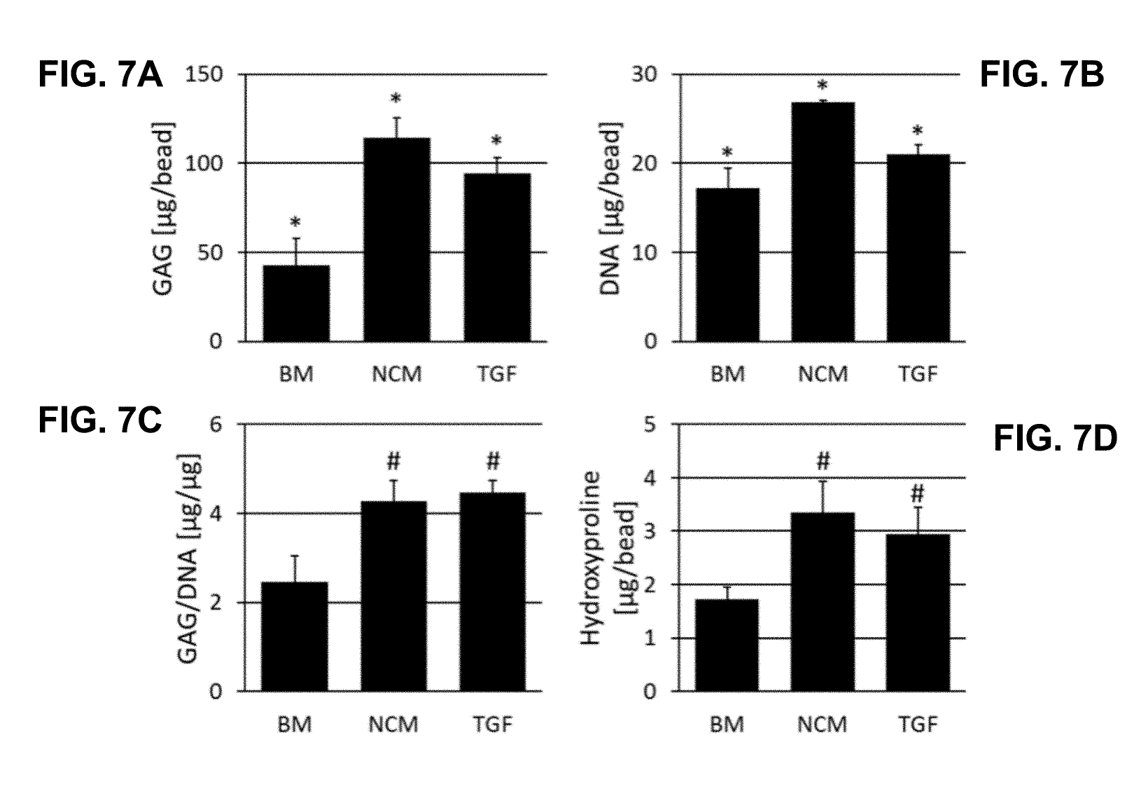

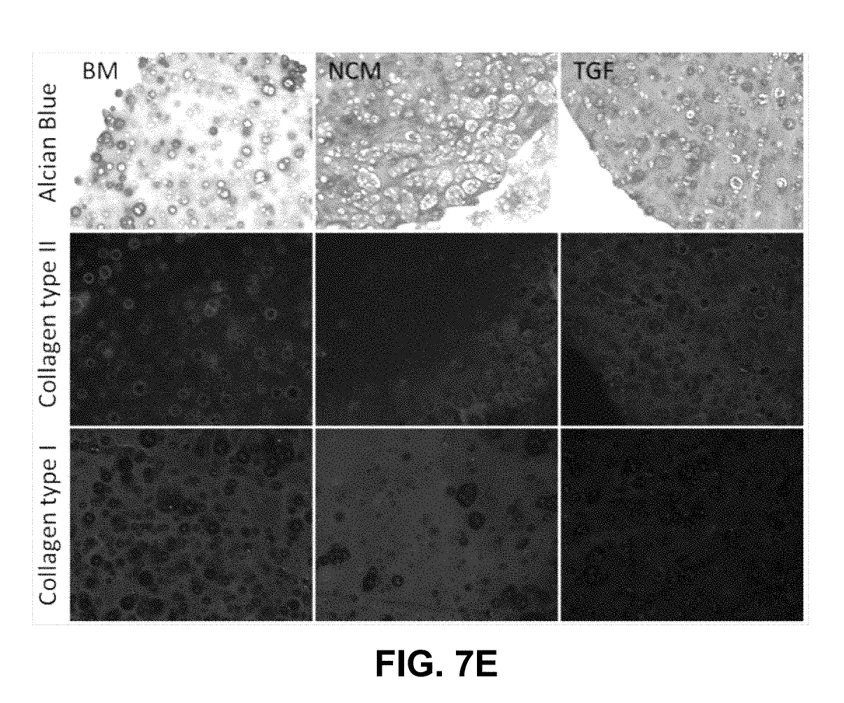

Both addition of NCM and TGF resulted in increased GAG content compared to BM (FIG. 7A). Furthermore, GAG content with NCM was significantly higher compared to TGF. A similar pattern is observed with DNA content, which increased with TGF compared to BM, but increased further with NCM (FIG. 7B). These data lead to a similar increased GAG per DNA ratio for NCM and TGF compared to BM (FIG. 7C). Also, hydroxyproline, as a measure for collagen content, increased with both NCM and TGF relative to BM (FIG. 7D). Alcian blue staining confirmed the increased GAG content with NCM and TGF (FIG. 7E). From collagen immunostainings, collagen type II deposition appeared to be stimulated with NCM mainly at the edge of the bead, but especially with TGF compared to BM. Collagen type I deposition appeared not to be affected by TGF, whereas beads cultured in NCM stained more intense.

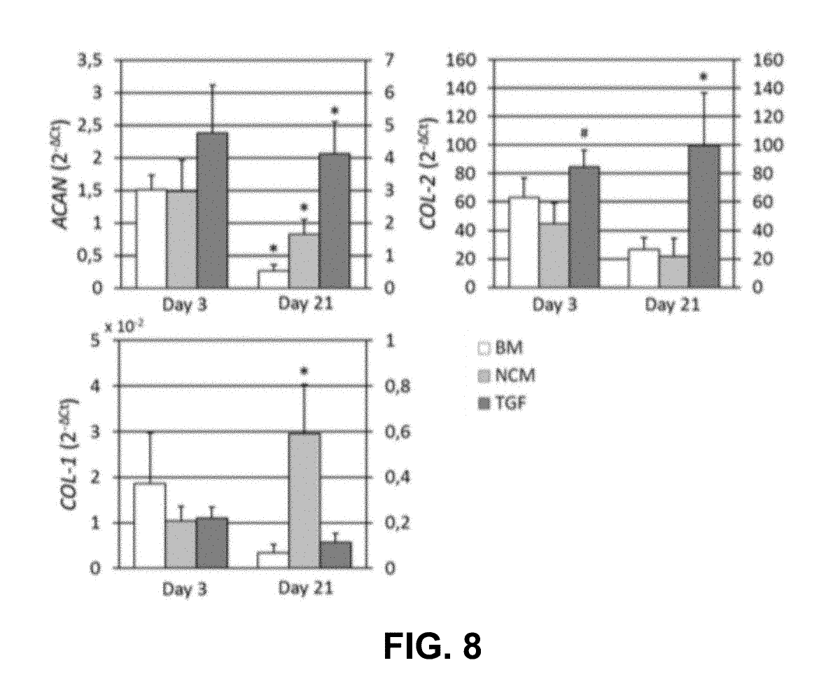

To determine the anabolic effect of NCM at the gene level, gene expression analysis of ACAN, COL-2 and COL-1 was performed (FIG. 8). At day 3 no differences in ACAN expression was observed between culture groups. At day 21 however, expression of ACAN increased with both NCM and TGF compared to BM, and with TGF compared to NCM. Expression of COL-2 was not significantly different with NCM compared to BM at day 3 and 21, but was significantly higher with TGF compared to NCM at day 3, and compared to BM and NCM at day 21. At day 3 no significant differences in COL-1 expression were observed between culture groups, however COL-1 expression was significantly higher in NCM compared to BM and TGF at day 21.

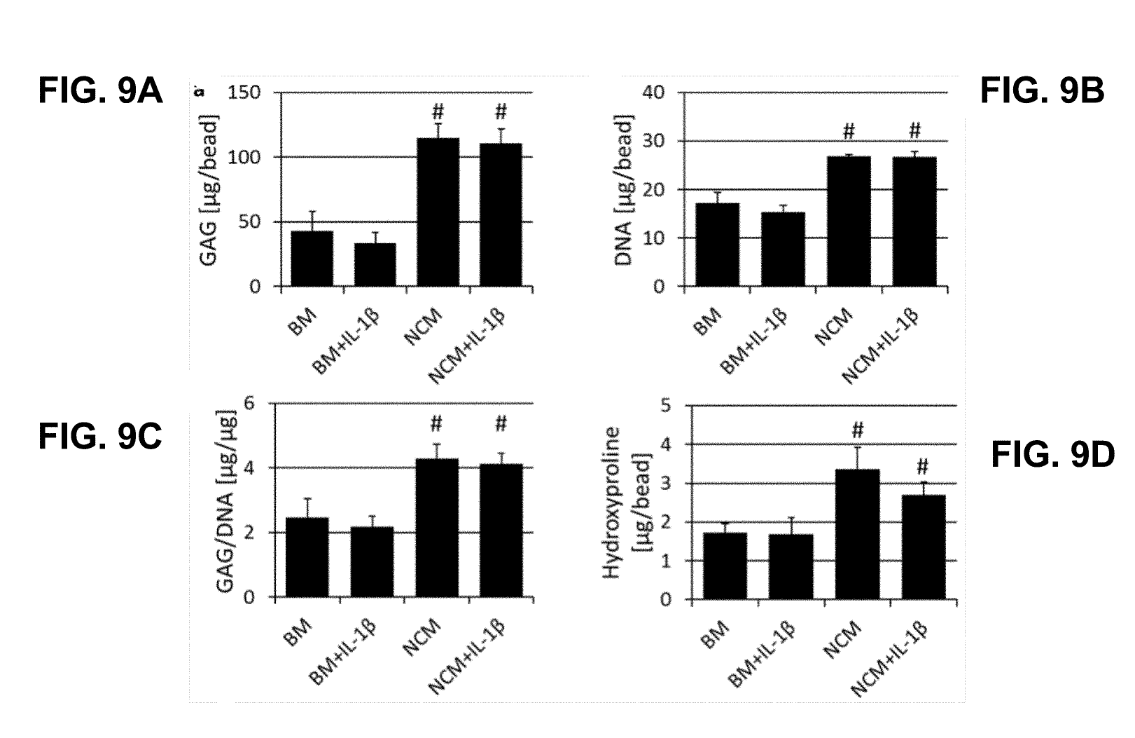

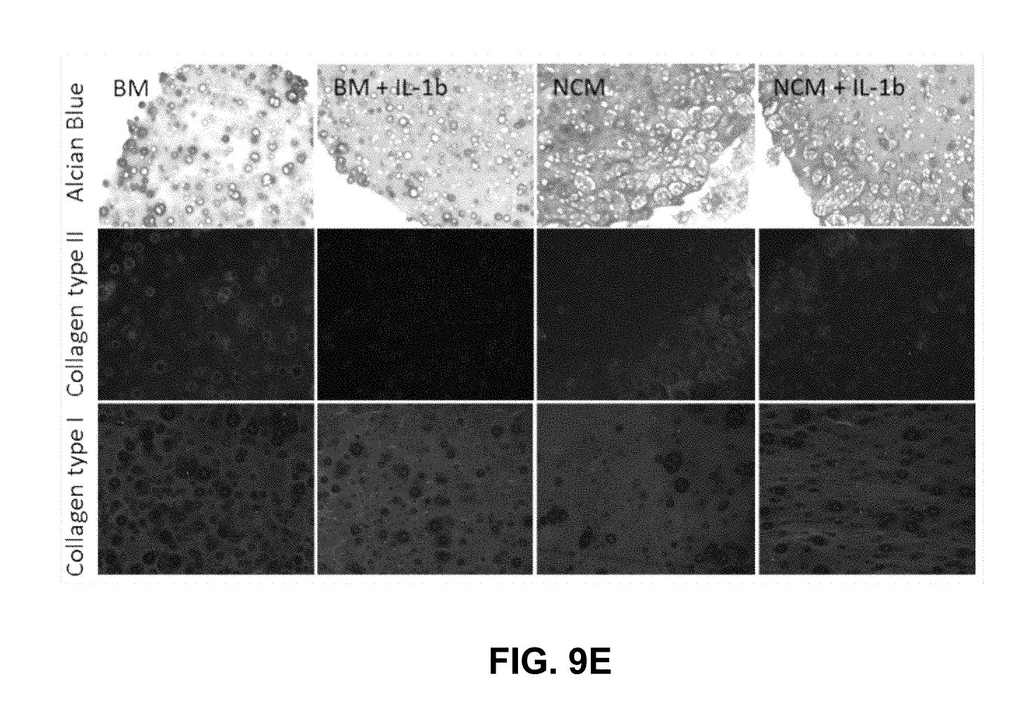

To determine whether NCM also has regenerative potential in the presence of an inflammatory stimulus, chondrocyte-seeded alginate beads were cultured in BM and NCM with and without addition of IL-1β. However, addition of IL-1β to BM and NCM did not affect GAG, DNA, GAG per DNA and hydroxyproline content compared to their counterparts without IL-1β (FIGS. 9aA-D). Alcian blue staining verified the increased GAG content with NCM with and without IL-1β compared to BM with and without IL-1β (FIG. 3e). Interestingly, immunostaining indicated that collagen type II is diminished with addition of IL-1β to BM, whereas this was not as clearly observed with addition of IL-1β to NCM. Furthermore, Addition of IL-1β appeared to increase the production of collagen type I in BM, but not in NCM.

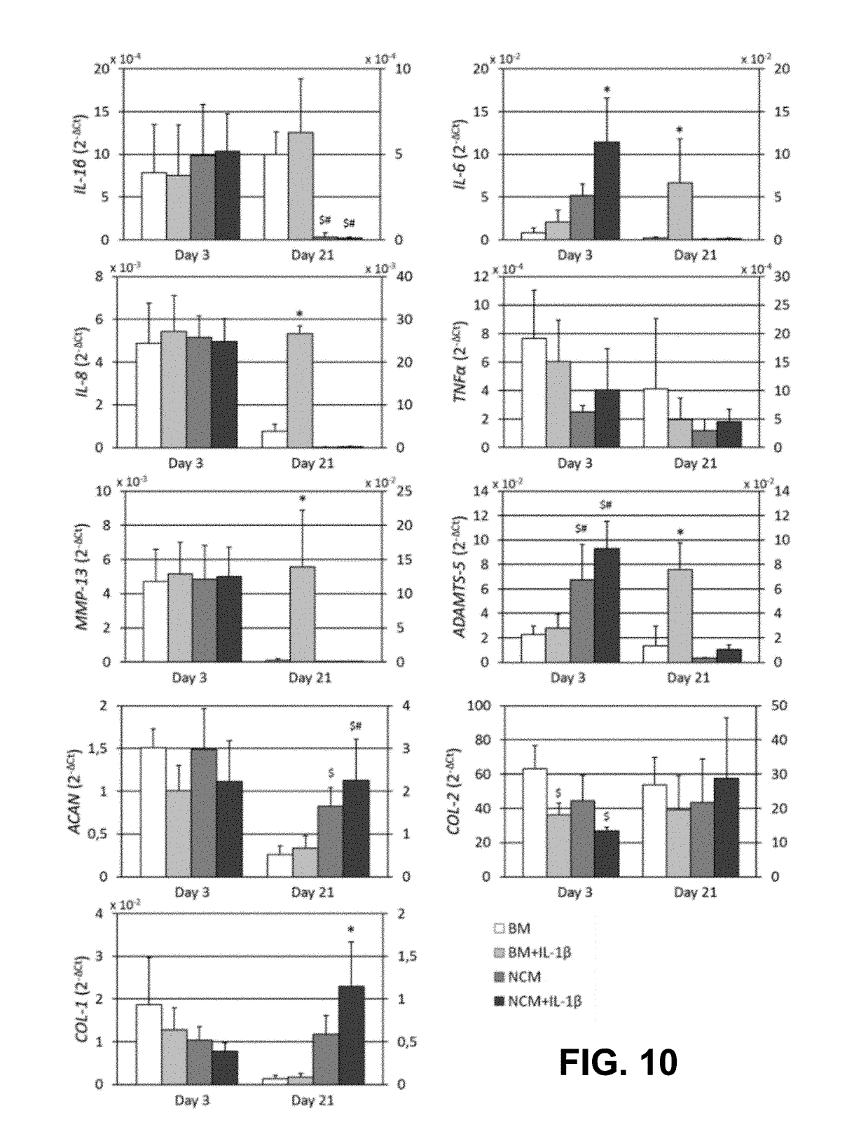

No differences in expression of IL-1β were observed between culture groups at day 3, whereas at day 21 IL-1β expression was significantly lower in both NCM groups compared to both BM groups (FIG. 10). Furthermore, addition of IL-1β did not increase IL-1β expression in either BM or NCM relative to their non-treated control. Expression of IL-6 was significantly higher in NCM with IL-1β compared to all other groups at day 3. However, at day 21 its expression was significantly higher to in BM with IL-1β compared to BM alone and both NCM groups. No differences in expression of IL-8 were observed at day 3, whereas its expression was significantly higher in BM with IL-1β compared to all other culture groups. No significant differences between culture groups were observed for TNFα at either day 3 or day 21. At day 3, no significant differences in expression of MMP-13 were observed, but its expression at day 21 was significantly higher in BM with IL-1β compared to BM alone and NCM groups. ADAMTS-5 expression at day 3 was significantly higher in NCM with and without IL-1β compared to both BM groups. However, at day 21 its expression was significantly higher in BM with IL-1β compared to the other groups. No differences in ACAN expression were observed at day 3. At day 21 however, ACAN expression in NCM was significantly higher compared to BM alone, and in NCM with IL-1β it was significantly higher compared to both BM groups. At day 3, addition of IL-1β significantly decreased COL-2 expression compared to BM alone, whereas no significant differences were observed at day 21. No differences were observed for COL-1 expression at day 3, but at day 21 its expression in NCM with IL-1β was significantly higher compared to all other culture groups.

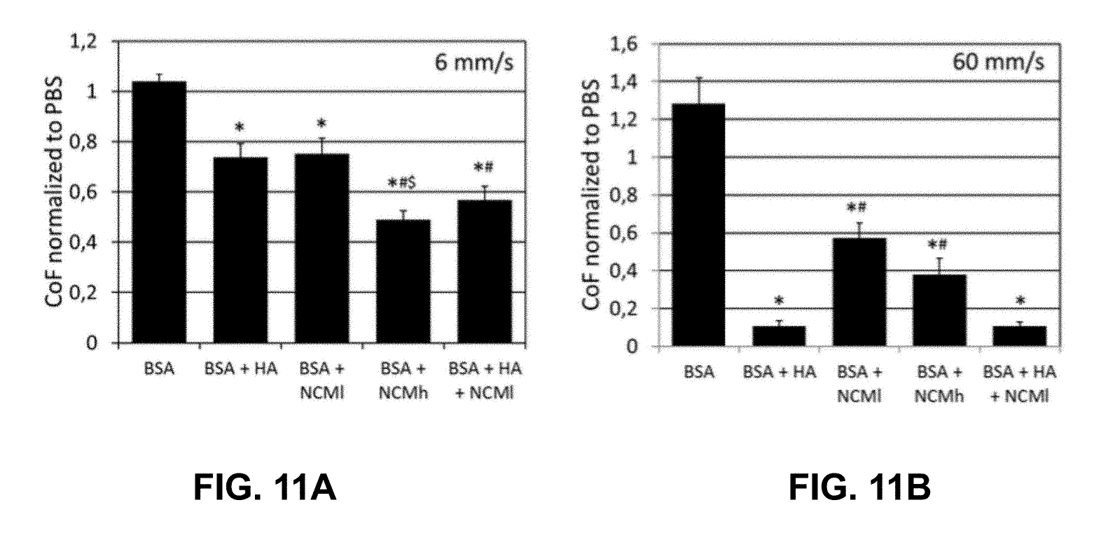

Towards NCM lubrication, at both 6 and 60 mm/s (FIGS. 11A-B) addition of BSA to PBS either made no difference or caused slight increase in CoF. At 6 mm/s, addition of HA and lower amounts of NCM (NCMl) resulted in a significant decrease (by ˜27%) in CoF after 20 cycles of sliding, compared to PBS with BSA (FIG. 11A). Combined addition of NCMl and HA resulted in a stronger reduction (45%) in CoF, which was significantly lower compared to BSA alone and BSA with HA, but not compared to BSA with NCMl. The strongest reduction (53%) in CoF was observed with addition of NCMh, where the CoF was significantly lower compared to BSA as well as both BSA with HA and BSA with NCMl.

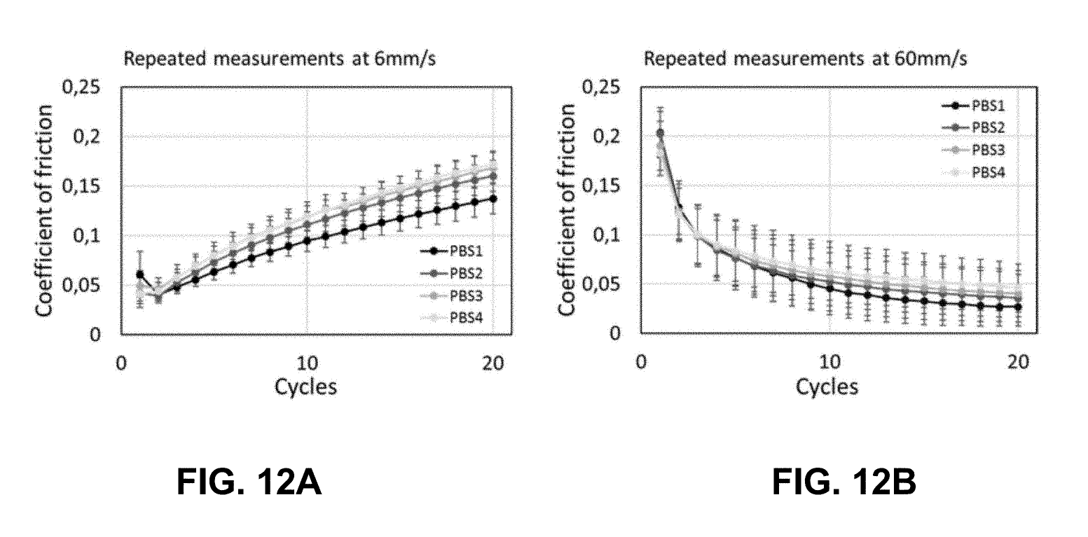

At 60 mm/s, addition of HA resulted in a 92% decrease in CoF after 20 cycles of sliding, compared to PBS with BSA, addition of HA and NCMl also showed a similar decrease. Addition of NCMl and NCMh respectively caused a significant decrease by 55 and 70% as compared to PBS with BSA (FIG. 11B). To verify that repeated sliding of the same plug did not affect CoF measurements, osteochondral plugs were slid against the glass surface for 4 rounds of 20 cycles, each round in fresh PBS. CoFs did not significantly change at either 6 (FIG. 12A) or 60 mm/s (FIG. 12B) as a result of multiple rounds of sliding. Therefore, no corrections were applied to the data presented in FIGS. 11A-B.

In conclusion, this embodiment demonstrates that NCM exerts regenerative effects on bovine chondrocytes, and has strong lubricating properties on articular cartilage. Therefore, NCM holds promise as a therapy for OA, where it may be applied to minimize pain directly upon injection into the joint, while simultaneously inducing a regenerative stimulus to the resident chondrocytes, that may restore the affected cartilage tissue towards a healthy state.

What is claimed is:

1. A method of making a notochordal cell matrix as a bioactive lubricant for an osteoarthritic joint, comprising:
(a) lyophilizing porcine nucleus pulposus tissue containing notochordal cells to destroy cells within the tissue and to make a dry and brittle tissue;
(b) treating the dry and brittle tissue to remove cellular and nucleic acid remnants, wherein the treatment results in at least 80% removal of porcine nucleic acids from the porcine nucleus pulposus tissue while substantially maintaining porcine protein content within the porcine nucleus pulposus tissue;
(c) lyophilizing the treated material and pulverizing the treated material into a notochordal cell matrix powder; and
(d) solubilizing the notochordal cell matrix powder by dissolving the notochordal cell matrix powder into a solution or a gel.

2. The method as set forth in claim 1, wherein the solubilized notochordal cell matrix powder is capable of stimulating native or stem cells to proliferate and produce a substantial increase in glycosaminoglycans and type-II collagen matrix.

3. The method as set forth in claim 1, wherein the solubilized notochordal cell matrix powder is capable of stimulating native or stem cells to proliferate and produce an at least a multi-fold increase in glycosaminoglycans.

4. A bioactive lubricant processed by the method as set forth in claim 1.

5. A bioactive lubricant, comprising: a solubilized notochordal cell matrix powder, wherein the notochordal cell matrix powder includes lyophilized and treated porcine nucleus pulposus tissue containing notochordal cells, wherein the powder contains less than 20% of porcine nucleic acids, wherein the powder contains a substantially unchanged amount of porcine protein content compared to the originating porcine nucleus pulposus tissue, and wherein the solubilized notochordal cell matrix powder is dissolved in a carrier solvent or formed as a gel.
